(19) United States
(12) Patent Application Publication
(10) Pub. No.: US 2024/0023825 A1
(43) Pub. Date: Jan. 25, 2024

(54) SYSTEM AND METHOD FOR DETERMINING A LEVEL OF OXYGEN CONSUMPTION OF A PATIENT

(71) Applicant: KONINKLIJKE PHILIPS N.V., Eindhoven (NL)

(72) Inventors: Roberto Buizza, Eindhoven (NL); Francesco Vicario, Cambridge, MA (US)

(21) Appl. No.: 18/142,231

(22) Filed: May 2, 2023

Related U.S. Application Data
(60) Provisional application No. 63/390,356, filed on Jul. 19, 2022.

(51) Int. Cl. A61B 5/083; A61B 5/08; A61B 5/097

(52) U.S. Cl. CPC A61B 5/0833; A61B 5/082; A61B 5/097

(57) ABSTRACT

According to an aspect, there is provided a system for determining a level of oxygen consumption of a patient, comprising: an inhalation limb configured to transport a first gas for inhalation by a patient; an exhalation limb configured to transport a second gas exhaled by the patient; a patient interface assembly connected to the inhalation limb and the exhalation limb, the patient interface assembly to transport the first gas to the patient and the second gas from the patient; a first oxygen sensor configured to measure a concentration of oxygen in the second gas at a first sampling region; a first carbon dioxide sensor configured to measure a concentration of carbon dioxide in the second gas at a second sampling region; a flow rate sensor configured to measure a flow rate of the first gas or the second gas; and a processor configured to determine a level of oxygen consumption of the patient using an indication of a concentration of oxygen in the first gas, an indication of a concentration of carbon dioxide in the first gas and data obtained via the first oxygen sensor, the first carbon dioxide sensor and the flow rate sensor.

### CROSS-REFERENCE TO RELATED APPLICATIONS

This patent application claims the priority benefit under 35 U.S.C. § 119(e) of U.S. Provisional Application No. 63/390,356, filed on Jul. 19, 2022, the contents of which are herein incorporated by reference.

### FIELD OF THE INVENTION

The invention relates to a system and method for determining a level of oxygen consumption of a patient. More particularly, the present invention relates to a system and method for determining a level of oxygen consumption of a patient using data received from various sensors.

### BACKGROUND OF THE INVENTION

Measurements allowing determination of an oxygen consumption (e.g., a volume of oxygen consumed, VO2) and a carbon dioxide production (e.g., a volume of carbon dioxide produced, VCO2) of a patient are highly desirable in a range of medical applications, including, for example, anaesthesia, critical care and nutrition management. Determining an oxygen consumption of a patient can be of particular value to clinicians as oxygen consumption can be indicative of a level of transport of oxygen from the patient's lungs to any of their tissues requiring oxygen. As such, oxygen consumption measurements can provide significant insights into the cardiovascular and metabolic status of a patient.

The size, cost, and accuracy of existing systems have limited the application of measurements allowing determination of oxygen consumption. Traditionally, expensive and bulky metabolic carts are used for these types of measurements, which require a long start-up time and are used intermittently.

More compact systems, which may be referred to as breath-by-breath systems, rely on high-frequency measurements of oxygen concentration within a gas flow. The accuracy of breath-by-breath systems is highly dependent on the precise time alignment of the measured oxygen concentration and gas flow waveforms.

Smaller, less costly and more accurate systems to determine oxygen consumption and carbon dioxide production are highly desirable.

### SUMMARY OF THE INVENTION

It is desirable to reduce the complexity and the cost associated with, and improve the accuracy of, determining oxygen consumption and carbon dioxide production of a patient. The inventors of the present disclosure have recognised that oxygen consumption and carbon dioxide production of a patient can be determined using a measurement of a concentration of oxygen in a gas exhaled by a patient at a first sampling location and using a commonly available measurement of a carbon dioxide concentration of the exhaled gas at a second sampling region. As a result, the architecture of devices required to determine an oxygen consumption of a patient is simplified.

More specifically, by determining a value of oxygen consumption of a patient using a measurement from a commonly available carbon dioxide sensor, such as a capnography device, the need for a separate, additional, carbon dioxide sensor as used in prior art systems is obviated. In addition, by using a commonly available carbon dioxide sensor, such as a capnography device, the rich clinical information of high-frequency volumetric capnography waveforms can be preserved.

According to a first specific aspect, there is provided a system for determining a level of oxygen consumption of a patient. The system comprises an inhalation limb configured to transport a first gas for inhalation by a patient, an exhalation limb configured to transport a second gas comprising gas exhaled by the patient, and a patient interface assembly connected to the inhalation limb and the exhalation limb. The patient interface assembly is to transport the first gas to the patient and the second gas from the patient. The system further comprises a first oxygen sensor configured to measure a concentration of oxygen in the second gas at a first sampling region, a first carbon dioxide sensor configured to measure a concentration of carbon dioxide in the second gas at a second sampling region, and a flow rate sensor configured to measure a flow rate of the first gas or the second gas. The system further comprises a processor configured to determine a level of oxygen consumption of the patient using an indication of a concentration of oxygen in the first gas, an indication of a concentration of carbon dioxide in the first gas, and data obtained via the first oxygen sensor, the first carbon dioxide sensor and the flow rate sensor. Advantageously, the need for a separate, additional, carbon dioxide sensor as used in prior art systems is obviated.

In some embodiments, the system may further comprise a first mixing chamber configured to receive a portion of the second gas. The first mixing chamber may comprise one or more of the first oxygen sensor and a second carbon dioxide sensor configured to measure a concentration of carbon dioxide in the portion of the second gas to be used for verification of the concentration of carbon dioxide measured using the first carbon dioxide sensor.

Advantageously, using a mixing chamber allows the second gas to mix thereby avoiding the need for high-frequency measurements of oxygen concentration requiring precise time alignment of the measured oxygen concentration and gas flow waveforms. Using a second carbon dioxide sensor to verify a measurement of the first carbon dioxide sensor may highlight when the first carbon dioxide sensor is faulty.

The first mixing chamber may, in some embodiments, be configured to receive a portion of the second gas via a connection with the patient interface assembly or via a connection with the exhalation limb.

In some embodiments, the system may further comprise a mechanical ventilator. The inhalation limb and the exhalation limb may be connected to the mechanical ventilator. One or more of the following conditions may be satisfied: the first mixing chamber may be configured to receive a portion of the second gas via a connection with an exhaust of the mechanical ventilator, the concentration of oxygen in the first gas may be determined using the mechanical ventilator, and the concentration of carbon dioxide in the first gas may be determined using the mechanical ventilator. Advantageously, by determining the concentration of oxygen and/or the concentration of carbon dioxide in the first gas using the mechanical ventilator, the need for additional sensors is obviated thereby simplifying the architecture of devices required to determine an oxygen consumption of a patient.

The system may, in some embodiments, further comprise one or more of a second oxygen sensor configured to measure the concentration of oxygen in the first gas at a third sampling region and a third carbon dioxide sensor configured to measure the concentration of carbon dioxide in the first gas at a fourth sampling region.

In some embodiments, the system may further comprise a second mixing chamber configured to receive a portion of the first gas via a connection with the inhalation limb or with the patient interface assembly, the second mixing chamber comprising one or more of the second oxygen sensor and the third carbon dioxide sensor.

The first carbon dioxide sensor may, in some embodiments, comprise a capnography device.

In some embodiments, the capnography device may be a mainstream capnography device or a sidestream capnography device.

The system may, in some embodiments, comprise a patient y-connector comprising a first port connected to an end of the inhalation limb, a second port connected to an end of the exhalation limb, and a third port connected to an end of the patient interface assembly.

According to a second aspect, a computer-implemented method for determining a level of oxygen consumption of a patient is provided. The method comprises receiving first oxygen concentration data indicative of a concentration of oxygen in a first gas to be inhaled by a patient. The method further comprises receiving first carbon dioxide data indicative of a concentration of carbon dioxide in the first gas. The method further comprises receiving second oxygen concentration data indicative of a concentration of oxygen in a second gas comprising gas exhaled by the patient, the concentration of oxygen of the second gas measured using an oxygen sensor at a first sampling region. The method further comprises receiving second carbon dioxide data indicative of a concentration of carbon dioxide in the second gas, the concentration of carbon dioxide in the second gas measured using a first carbon dioxide sensor at a second sampling region. The method further comprises receiving flow rate data indicative of a flow rate of the first gas or the second gas. The method further comprises determining, based on the received data, a level of oxygen consumption of the patient.

In some embodiments, the method may further comprise receiving third carbon dioxide data indicative of a concentration of carbon dioxide in the second gas measured using a second carbon dioxide sensor located at the first sampling location, comparing the second carbon dioxide data with the third carbon dioxide data, and generating an alert responsive to determining that a difference between the second carbon dioxide data and the third carbon dioxide data exceeds a threshold level.

Determining a level of oxygen consumption, VO2, of the patient may comprise using the formula

$$VO 2 = {V}_{exh} ( FiO 2 * \frac{( 1 - FeO 2 - FeCO 2 )}{( 1 - FiO 2 - FiCO 2 )} - FeO 2 ) or \\ VO 2 = {V}_{inh} ( FiO 2 - FeO 2 * \frac{( 1 - FiO 2 - FiCO 2 )}{( 1 - FeO 2 - FeCO 2 )} ) .$$

FiO2 is a concentration of oxygen in the first gas, FiCO2 is a concentration of carbon dioxide in the first gas, FeO2 is a concentration of oxygen in the second gas, FeCO2 is a concentration of carbon dioxide in the second gas, Vₑₓₕ is the average flow rate of the second gas at the point of measurement of the concentration of oxygen in the second gas, and Vᵢₙₕ is the average flow rate of the first gas. FeCO2 is determined using the formula

$$\frac{VCO 2}{{V}_{exh}} or \frac{VCO 2}{{V}_{inh}} ,$$

wherein VCO2 is a value of carbon dioxide production determined using the second carbon dioxide data. Using only one flow rate measurement, as opposed to two flow rate measurements, reduces the error in the determination of oxygen consumption of a patient.

In some embodiments, each of the variables Vₑₓₕ, FiO2, FeO2, FiCO2 and FeCO2 may represent an average value over a defined time period.

First carbon dioxide data may, in some embodiments, comprise an ambient carbon dioxide concentration. Advantageously, this feature leads to a further simplification of the architecture of devices required to determine an oxygen consumption of a patient.

According to a third aspect, a computer program product may be provided. The computer program product may comprise a non-transitory computer readable medium, the computer readable medium having computer readable code embodied therein, the computer readable code being configured such that, on execution by a suitable computer or processor, the computer or processor is caused to perform steps of the methods described herein.

These and other aspects will be apparent from and elucidated with reference to the embodiment(s) described hereinafter.

### BRIEF DESCRIPTION OF THE DRAWINGS

Exemplary embodiments will now be described, by way of example only, with reference to the following drawings, in which:

FIG. 1 is a schematic illustration of an example of a system for determining a level of oxygen consumption of a patient;

FIG. 2 is a schematic illustration of a further example of a system for determining a level of oxygen consumption of a patient;

FIG. 3 is a flowchart of an example of a computer-implemented method for determining a level of oxygen consumption of a patient;

FIG. 4 is a flowchart of a further example of a computer-implemented method for determining a level of oxygen consumption of a patient; and

FIG. 5 is a schematic illustration of a processor in communication with a non-transitory computer readable medium.

### DETAILED DESCRIPTION OF EMBODIMENTS

Determining levels of oxygen consumption and carbon dioxide production of a patient may provide an insight into the subject's health and well-being. For example, measurements allowing determination of levels of oxygen consumption and carbon dioxide production of a patient may be referred to as metabolic monitoring, since these parameters allow an amount of energy expenditure of the body of a patient to be inferred (e.g., indirect calorimetry). Also, oxygen consumption data can be indicative of a level of transport of oxygen from the patient's lungs to regions of their tissue requiring oxygen and can therefore be of particular importance when monitoring critically ill patients. As such, oxygen consumption data can provide significant insights into the cardiovascular and metabolic status of a patient.

Oxygen consumption may be defined as the difference between an inhaled volume of oxygen, VᵢₙₕO2, and an exhaled volume of oxygen, VₑₓₕO2, and can be determined using the following equation:

VO2=Vᵢₙₕ*FiO2−Vₑₓₕ*FeO2 (1)

The inhaled and exhaled volumes of oxygen may be computed by direct measurement or indirect estimation. In some examples, Vₑₓₕ may be measured directly whereas Vᵢₙₕ may be inferred indirectly using, for example, the Haldane transformation. In the Haldane transformation, a volume of gas inhaled by a patient, Vᵢₙₕ, is estimated from a volume of gas exhaled by the patient, Vₑₓₕ. The Haldane transformation is based on the assumption that the volumes of inhaled and exhaled nitrogen (an inert gas) are equal. The following equation allows the computation of the inhalation volume to be computed:

$${V}_{inh} = {V}_{exh} \frac{( 1 - FeO 2 - FeCO 2 )}{( 1 - FiO 2 - FiCO 2 )} \qquad ( 2 )$$

where FiO2 is the fraction of oxygen in a gas to be inhaled by a patient, FiCO2 is the fraction of carbon dioxide in the gas to be inhaled by the patient, FeO2 is the fraction of oxygen in a gas exhaled by the patient and FeCO2 is the fraction of carbon dioxide in the gas exhaled by the patient. FiO2 may refer to a concentration of oxygen in a gas to be inhaled by a patient. FiCO2 may refer to a concentration of carbon dioxide in a gas to be inhaled by a patient. A gas to be inhaled by a patient may refer to a sample of gas to be delivered to, and/or inhaled by, the patient. FeO2 may refer to a concentration of oxygen in a gas exhaled by a patient. FeCO2 may refer to a concentration of carbon dioxide in a gas exhaled by a patient. A gas exhaled by a patient may refer to a sample of gas exhaled by the patient. Similarly, the following equation allows the computation of the exhalation volume to be calculated based on a value (e.g., a measurement) of the inhalation volume:

$${V}_{exh} = {V}_{inh} \frac{( 1 - FiO 2 - FiCO 2 )}{( 1 - FeO 2 - FeCO 2 )} \qquad ( 3 )$$

In some examples, a patient may breathe in a gas received from a mechanical ventilator. For example, the patient may be connected to an endotracheal tube or a face mask configured to supply the patient with a flow of gas from a mechanical ventilator. The oxygen concentration associated with the gas flow received from a mechanical ventilator may be set to a defined value, such as 20%, 25%, 30%, 40%, 50%, 60% by volume, or the like. The FiO2 value used in equations 4 and 5 (and similarly equations 6 and 7) to determine oxygen consumption and carbon dioxide production, respectively, may be the oxygen concentration that the mechanical ventilator is set to. In some examples, the value of oxygen concentration in an inhaled gas (e.g., as supplied by the mechanical ventilator) may be assumed to be constant during the course of an inhalation (e.g., an inhaled breath) by a patient. In some examples, an FiO2 value may be determined using an oxygen sensor (e.g., an oxygen sensor located within the mechanical ventilator).

In some examples, an FiCO2 value may be determined using the mechanical ventilator (e.g., a carbon dioxide sensor located in the mechanical ventilator). In other examples, an FiCO2 value may be assumed to be the ambient carbon dioxide concentration of air in the atmosphere (e.g., 400 ppm). An FiCO2 value may, in some examples, be set to a value of zero in light of the typically relatively higher concentration of carbon dioxide in a gas exhaled by a patient. In some examples, an FiCO2 value may be measured using a carbon dioxide sensor configured to sample gas, or a sample of gas, to be inhaled by the patient.

In some examples, an FeO2 value may be determined using an oxygen sensor configured to sample gas, or a sample of gas, exhaled by the patient. An FeCO2 value may be determined using a carbon dioxide sensor configured to sample gas, or a sample of gas, exhaled by the patient.

The exhaled volume, Vₑₓₕ, may be determined by measuring gas flow during the exhalation phase. The exhaled volume, Vₑₓₕ, may be determined using the flow rate of the exhaled gas, Qₑₓₕ. In some examples, Vₑₓₕ may refer to an average flow (e.g., flow rate) of gas exhaled by a patient (e.g., the average flow rate per minute). Using a mixing chamber to measure a concentration of gas may allow an average value of Vₑₓₕ to be used. An advantage of using equation 2 to determine a volume of gas inhaled by a patient (or equation 3 to determine a volume of gas exhaled by a patient) is that only a single flow rate measurement (e.g., from a single flow rate sensor) is required to estimate oxygen consumption and carbon dioxide production, thereby reducing the contribution of flow measurements errors to the overall error in the determination of oxygen consumption.

Oxygen consumption and carbon dioxide production can be determined by combining equations 1 and 2:

$$\begin{matrix}VO 2 = {V}_{exh} ( FiO 2 * \frac{( 1 - FeO 2 - FeCO 2 )}{( 1 - FiO 2 - FiCO 2 )} - FeO 2 ) & ( 4 ) \\ VCO 2 = {V}_{exh} ( FeCO 2 - FiCO 2 * \frac{( 1 - FeO 2 - FeCO 2 )}{( 1 - FiO 2 - FiCO 2 )} ) & ( 5 )\end{matrix}$$

Equations 4 and 5 may provide the oxygen consumption and carbon dioxide production, respectively, based on values (e.g., measurements) of Vₑₓₕ, FiO2, FeO2, FiCO2 and FeCO2. For example, measurements of Vₑₓₕ, FiO2, FeO2, FiCO2 may be taken every 0.1 seconds, 1 second, 2 second, 5 seconds, or the like. These measurements may be averaged over a timescale of 1 second, 2 seconds, 5 seconds, 10 seconds, 1 minute, or the like. An advantage associated with averaging measurements of Vₑₓₕ, FiO2, FeO2, FiCO2 is that the signal-to-noise ratio associated with the measurements may be increased. A further advantage of averaging measurements of Vₑₓₕ, FiO2, FeO2, FiCO2 is that time-alignment of each of the measurements may be less critical, less time consuming, and less prone to misalignments. Therefore, the accuracy with which oxygen production and carbon dioxide production can be determined may be improved. These advantages may be further realised by use of a mixing chamber to sample a gas (e.g., a gas to be inhaled by a patient or a gas comprising gas exhaled by a patient), as is explained in greater detail below.

Oxygen consumption and carbon dioxide production can also be determined by combining equations 1 and 3:

$$\begin{matrix}VO 2 = {V}_{inh} ( FiO 2 - FeO 2 * \frac{( 1 - FiO 2 - FiCO 2 )}{( 1 - FeO 2 - FeCO 2 )} ) & ( 6 ) \\ VCO 2 = {V}_{inh} ( FiCO 2 - FeCO 2 * \frac{( 1 - FiO 2 - FiCO 2 )}{( 1 - FeO 2 - FeCO 2 )} ) & ( 7 )\end{matrix}$$

The decision to use the inhalation or exhalation flow sensor may depend on which position will provide more accurate flow measurements.

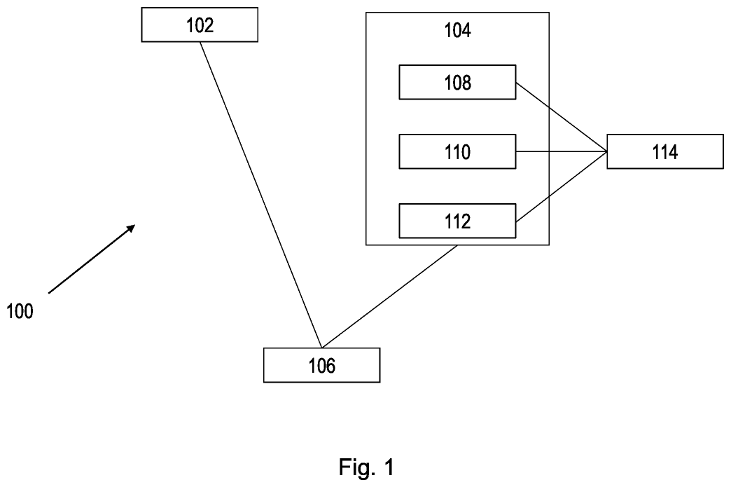

To determine an accurate value of oxygen consumption, measurements of Qₑₓₕ, and thus Vₑₓₕ, may be taken at a point that is representative of the flow rate, and thus volume, of exhaled gas at a point at which the oxygen concentration measurement of the exhaled gas is taken. This feature is explained in more detail with reference to FIG. 1 below. Similarly, measurements of Qᵢₙₕ, and thus Vᵢₙₕ, may be taken at a point that is representative of the flow rate, and thus volume, of inhaled gas at a point corresponding to the point at which the oxygen concentration measurement of the exhaled gas is taken. This will be explained in more detail with reference to FIG. 1 hereinafter.

According to a first aspect, a system for determining a level of oxygen consumption of a patient is provided. FIG. 1 is a schematic illustration of an example of a system 100 for determining a level of oxygen consumption of a patient. The system 100 comprises an inhalation limb 102 configured to transport a first gas for inhalation by a patient. In some embodiments, the system 100 may comprise a mechanical ventilator (not shown in FIG. 1). The inhalation limb may comprise a tube. For example, the inhalation limb may comprise a tube connected at a first end to a mechanical ventilator and connected at a second end to a patient interface assembly. The first gas may comprise one or more gases, such as oxygen, nitrogen, carbon dioxide, or the like. The first gas may comprise oxygen at a concentration determined by a setting of the ventilator, such as 20%, 30%, 50%, or the like. A gas pressure of the first gas may be ambient pressure (e.g., 1 bar) or may be at an elevated pressure (e.g., higher than ambient pressure). The first gas may be at a pressure of, or in excess of, 5 cmH20, 10 cmH20, 15 cmH20, 20 cmH20, 30 cmH20, or the like. The first gas being at a pressure higher than ambient pressure may facilitate assisted breathing of the patient and/or may cause the first gas to flow along the inhalation limb 102 in a direction from the mechanical ventilator towards the patient. In some examples, the mechanical ventilator may comprise a pump to cause the first gas to flow along the inhalation limb in a direction from the mechanical ventilator towards the patient.

The system 100 further comprises an exhalation limb 104 configured to transport a second gas comprising gas exhaled by the patient. The second gas may comprise gas exhaled by a patient, or a mixture of gas exhaled by a patient and the first gas (e.g., surplus first gas not required by the patient for breathing). The exhalation limb 104 may comprise a tube. For example, the exhalation limb 104 may comprise a tube connected at a first end to the mechanical ventilator and connected at a second end to the patient interface assembly. The second gas may comprise oxygen, carbon dioxide, nitrogen, water vapour, or the like (e.g., any gases exhaled by the patient).

The system 100 further comprises a patient interface assembly 106 connected to the inhalation limb 102 and the exhalation limb 104, the patient interface assembly 106 configured to transport the first gas to the patient and the second gas from the patient. In some examples, the patient interface assembly 106 may comprise an endotracheal tube or a face mask. In some examples, the patient interface assembly 106 may be connected to a tube configured to transport the first gas to the patient and the second gas from the patient. For example, the tube may be connected at a first end to the patient interface assembly 106 and at a second end to an endotracheal tube or a face mask. In some examples, the patient interface assembly 106 may comprise a Y-piece, or a Y-connector, comprising three ports (e.g., inlets or outlets) connected to the inhalation limb 102, the exhalation limb 104 and an end of the patient interface assembly, respectively. In some examples, the Y-connector may be connected to the inhalation limb 102, the exhalation limb 104 and the tube, respectively, wherein the tube may be connected to the patient interface assembly 106. A Y-connector may be referred to as a patient wye or a patient wye connector.

In some examples, the volume of the first gas supplied to the patient via the inhalation limb 102 is greater than the volume of gas needed for breathing. In some examples, any excess first gas (e.g., surplus first gas not required by the patient for breathing) may flow from the inhalation limb 102 to the exhalation limb 104 (e.g., using the patient interface assembly 106). In some examples, the excess first gas may be referred to as a bypass, or bias, flow.

The system 100 further comprises a first oxygen sensor 108 configured to measure a concentration of oxygen in the second gas at a first sampling region. In some examples, the oxygen sensor 108 may measure a concentration of oxygen in the second gas via a side-stream connection. For example, a portion of the second gas travelling along the exhalation limb 104 may be separated from the main flow of the second gas, and the oxygen sensor 108 may be configured to measure a concentration of oxygen in the portion of the second gas. In some examples, the portion of the second gas may be introduced back into the main flow of the second gas after the oxygen sensor 108 has measured a concentration of oxygen of the portion of the second gas. In some examples, the oxygen sensor 108 may be located within a mixing chamber configured to receive the second gas, as described in more detail below.

In some examples, the first sampling region may comprise a volume configured to sample the second gas via a side-stream connection. In some examples, the first sampling region may be located at a point along the exhalation limb 104, at a point within the patient interface assembly 106 (e.g., at a port of the patient interface assembly), at the exhaust of a mechanical ventilator, or the like. If the first sampling region is located at a port of the patient interface assembly 106, then the oxygen sensor may be configured to measure a concentration of oxygen only during an exhalation phase of the patient's breathing. In some examples, the first sampling region may comprise a region (e.g., a volume, a mixing chamber, or the like) configured to sample a portion of the second gas. In some examples, the first sampling region may comprise a chamber or enclosure that receives a sample (e.g., a portion) of the second gas out of the main flow of the second gas (e.g., travelling through the patient interface assembly 106 or the exhalation limb 104).

The system 100 further comprises a first carbon dioxide sensor 110 configured to measure a concentration of carbon dioxide in the second gas at a second sampling region. In some examples, the second sampling region may be located at a port of the patient interface assembly 106. In some examples, the first carbon dioxide sensor 110 may comprise a capnography device (e.g., a Capnostat). In some examples, the first carbon dioxide sensor 110 may form a part of, or be a component of, a capnography device. In some examples, the second sampling region may comprise a sampling region (e.g., a volume, enclosure, or the like) comprising a carbon dioxide sensor. The second sampling region may comprise a sampling region (e.g., a volume, a mixing chamber, or the like) configured to receive a portion of the second gas. In some examples, the second sampling region may comprise a different volume to the first sampling region (e.g., the first sampling region may comprise a volume within a first mixing chamber and the second sampling region may comprise a volume within a second mixing chamber). For example, a mixing chamber may comprise the first sampling region and a capnography device may comprise the second sampling region. In some examples, a carbon dioxide production value may be determined based on a measurement of a concentration of carbon dioxide measured using the first carbon dioxide sensor 110. In some examples, a processor 114 associated with a carbon dioxide sensor (e.g., a capnography device) may be configured to determine a carbon dioxide production value based on a measurement of a concentration of carbon dioxide measured using the first carbon dioxide sensor 110. A capnography device may be able to measure a carbon dioxide production value to an accuracy of ±10%. Measuring a concentration of carbon dioxide in the second gas using a capnography device may preserve rich clinical information associated with high-frequency volumetric capnography waveforms measured at a port of the patient interface assembly 106. If a capnography device is used to measure a concentration of carbon dioxide in the second gas via a port of the patient interface assembly 106, it may be possible to determine clinical measurements including, for example, end-tidal carbon dioxide and dead space.

In some examples, a carbon dioxide production value determined using a measurement of a concentration of carbon dioxide of the second gas at the second sampling location using the first carbon dioxide sensor 110 may be used to determine a value of a concentration of carbon dioxide in the second gas at the first sampling location. For example, the following equation may be used to determine a concentration of carbon dioxide in a gas based on a carbon dioxide production:

$$FeCO 2 = \frac{VCO 2}{{V}_{exh}} \qquad ( 8 )$$

To determine a level of oxygen consumption of a patient accurately, the value of Vₑₓₕ may correspond to the flow rate of the second gas at the point of measurement of a concentration of oxygen of the second gas measured by the first oxygen sensor 108 (e.g., the first sampling location). Equation 8 may be applicable irrespective of where the VCO2 measurement is performed (e.g., the second sampling location). For example, where a concentration of oxygen of the second gas is measured by an oxygen sensor via a connection with the patient interface assembly 106, then a flow rate measurement of the second gas travelling through the patient interface assembly may be used in equation 8. In examples where a concentration of oxygen of the second gas is measured by an oxygen sensor via a connection with the exhalation limb 104, then a flow rate measurement of the second gas travelling through the exhalation limb 104 may be used in equation 8. Using a measurement of the flow rate of the second gas at the point of measurement of a measurement of the oxygen concentration of the second gas ensures that the flow rate of any additional (e.g., excess first gas or bypass gas) that may be diluting the concentration of carbon dioxide in the second gas is taken account of.

In other words, equation 8 may be used to estimate a concentration of carbon dioxide in the second gas in the exhalation limb 104 (e.g., the first sampling location) using a value of carbon dioxide production of a patient determined using a measurement of a concentration of carbon dioxide in the patient interface assembly 106 (e.g., the second sampling location). Estimating a concentration of carbon dioxide in the second gas in the exhalation limb 104 in this way (e.g., using a carbon dioxide sensor at the second sampling location) may obviate the need for a carbon dioxide sensor (e.g., an additional carbon dioxide sensor) configured to measure a concentration of carbon dioxide in the second gas at the first sampling location.

In embodiments where a flow rate measurement is being taken on the inhalation side (e.g., the inhalation limb), then Vₑₓₕ in equation 8 may be assumed to be Vᵢₙₕ. In other words, Vₑₓₕ may be replaced with Vᵢₙₕ.

In some embodiments, a mainstream capnography device may be used. A mainstream capnography device is a device that measures a concentration of carbon dioxide in-situ (e.g., using a carbon dioxide sensor located directly in the flow of the second gas). In some embodiments, a side-stream capnography device may be used (e.g., using a carbon dioxide sensor configured to sample a portion of the second gas separated from the main flow of the second gas). In other words, a portion of the second gas may be syphoned off from, or tapped from, the main flow of the second gas travelling along the inhalation limb 102.

Furthermore, in clinical environments, capnography devices are commonly used, for example, to monitor mechanically ventilated patients. Advantageously, measuring a concentration of carbon dioxide in the second gas using a capnography device, as opposed to using a separate carbon dioxide sensor located at a point along the exhalation limb 104, simplifies the design of the system (e.g., a carbon dioxide sensor located on the exhalation limb 104 may not be required). In other words, in prior art systems, a carbon dioxide sensor would be required on the exhalation limb 104 (e.g., in addition to a capnography device), while in the system disclosed herein, the need for such a carbon dioxide sensor is obviated. Furthermore, using a carbon dioxide sensor, such as a capnography device, to sample the exhaled gas directly preserves the rich clinical information associated with high-frequency volumetric capnography waveforms. This information may be lost if a measurement of a concentration of carbon dioxide of the second gas is performed by a carbon dioxide sensor located within a mixing chamber configured to sample a portion of the second gas.

The system 100 further comprises a flow rate sensor 112 configured to measure a flow rate of the first gas or the second gas. In some examples, the flow rate sensor 112 may be configured to measure a flow rate of the first gas at a point along the inhalation limb. In other examples, the flow rate sensor 112 may be configured to measure a flow rate of the first gas passing through the patient interface assembly 106 (e.g., during the inhalation phase of the patient's breathing). In some examples, the flow rate sensor 112 may be configured to measure a flow rate of the second gas passing through the patient interface assembly 106 (e.g., during the exhalation phase of the patient's breathing). In some examples, the flow rate sensor 112 may be configured to measure a flow rate of the second gas at a point along the exhalation limb 104, at the exhaust of the mechanical ventilator, or the like.

The system 100 further comprises a processor 114 configured to determine a level of oxygen consumption of the patient using an indication of a concentration of oxygen in the first gas, an indication of a concentration of carbon dioxide in the first gas and data obtained via the first oxygen sensor 108, the first carbon dioxide sensor 110 and the flow rate sensor 112. In some examples, the processor 114 is configured to determine a level of oxygen consumption of the patient by implementing equation 4 (or equation 6). In some examples, the processor 114 may be configured to determine a level of carbon dioxide production of the patient using an indication of a concentration of oxygen in the first gas, an indication of a concentration of carbon dioxide in the first gas and data obtained via the first oxygen sensor 108, the first carbon dioxide sensor 110 and the flow rate sensor 112 (e.g., by implementing equation 5). In some examples, the processor 114 may be configured to determine a level of energy expenditure of the body of the patient based on the determined oxygen consumption and the carbon dioxide production.

In some embodiments, the system 100 may comprise a first mixing chamber configured to receive a portion of the second gas. The first mixing chamber may comprise the first oxygen sensor 108 and/or a second carbon dioxide sensor configured to measure a concentration of carbon dioxide in the portion of the second gas to be used for verification of the concentration of carbon dioxide measured using the first carbon dioxide sensor 110. In some examples, the first mixing chamber may be configured to receive a portion of the second gas via a connection with the patient interface assembly 106 (e.g., a port of the patient interface assembly). In other examples, the first mixing chamber may be configured to receive a portion of the second gas via a connection with a port of the exhalation limb 104. In some examples, the first mixing chamber may be configured to receive a portion of the second gas via a connection with the exhaust of a mechanical ventilator. In some examples, the first mixing chamber may be located in-line with the patient interface assembly 106, the exhalation limb 104, or the exhaust of the mechanical ventilator. For example, the first mixing chamber may be configured such that all of the second gas flows through the first mixing chamber.

In some embodiments, the system 100 may comprise a mechanical ventilator. The inhalation limb 102 and the exhalation limb 104 may be connected to the mechanical ventilator. In some examples, the first mixing chamber may be configured to receive a portion of the second gas via a connection with an exhaust of the mechanical ventilator. In some examples, the concentration of oxygen in the first gas may be determined using the mechanical ventilator and/or the concentration of carbon dioxide in the first gas may be determined using the mechanical ventilator. In some examples, the indication of a concentration of oxygen in the first gas, used by the processor 114 of system 100 to determine a level of oxygen consumption of the patient, may be received from the mechanical ventilator. In some examples, the indication of a concentration of carbon dioxide in the first gas, used by the processor 114 of system 100 to determine a level of oxygen consumption of the patient, may be received from the mechanical ventilator.

In some embodiments, the system 100 may comprise a second oxygen sensor configured to measure the concentration of oxygen in the first gas at a third sampling region. The third sampling region may be located at a point along the inhalation limb 102 or at a point along the patient interface assembly 106 (e.g., at a port of the patient interface assembly). Measuring the oxygen concentration in this way may lead to a more accurate determination of oxygen consumption (e.g. rather than relying on an oxygen concentration value received from the mechanical ventilator). In some examples, the system may comprise a third carbon dioxide sensor configured to measure the concentration of carbon dioxide in the first gas at a fourth sampling region. The fourth sampling region may be located at a point along the inhalation limb 102 or at a point along the patient interface assembly 106 (e.g., at a port of the patient interface assembly).

In some embodiments, the system 100 may comprise a second mixing chamber configured to receive a portion of the first gas via a connection with the inhalation limb 102 or with the patient interface assembly 106. The second mixing chamber may comprise the second oxygen sensor and/or the third carbon dioxide sensor. In some examples, the third sampling region and the fourth sampling region may be located within the second mixing chamber.

As mentioned previously, to determine an accurate value of oxygen consumption, measurements of Qₑₓₕ, and thus Vₑₓₕ, may be taken at a point that is representative of the flow rate, and thus volume, of exhaled gas at a point at which the oxygen concentration measurement of the exhaled gas is taken. With reference to FIG. 1, if the first oxygen sensor 108 is configured to sample a portion of the second gas exhaled by the patient via a connection to the exhalation limb 104, then the flow rate sensor 112 may be configured to sample a portion of the second gas via a connection to the exhalation limb 104. The flow rate sensor 112 may be configured to measure the flow rate of the second gas (e.g., the entire flow, or the main flow, of the second gas). For example, the flow rate sensor may be configured to measure the flow rate of gas in the exhalation limb or in the patient interface assembly. The first oxygen sensor 108 may be located upstream or downstream of the flow rate sensor 112. The precise points along the exhalation limb 104 in which the first oxygen sensor 108 and the flow rate sensor 112 are connected, respectively, may not be critical, as the flow rate of the second gas along the exhalation limb 104 may not change.

In some examples, the first oxygen sensor 108 may be configured to sample a portion of the second gas via a connection with the patient interface assembly 106. In this case, a flow rate measurement may be configured to sample a portion of the second gas via a connection with the patient interface assembly 106. The first oxygen sensor 108 and/or the flow rate sensor 112 may be configured to sample gas only during the exhalation phase of the patient's breathing. In some examples, the first oxygen sensor 108 and/or the flow rate sensor 112 may be located in a mixing chamber connected to the patient interface assembly 106. In some examples, the first oxygen sensor 108 and/or the flow rate sensor 112 may be connected to the patient interface assembly 106 via a connection with the patient interface assembly. The precise location in which the first oxygen sensor 108 and/or the flow rate sensor 112 are configured to sample a portion of the second gas from the patient interface assembly 106 may not be critical, as the flow rate of exhaled gas through the patient interface assembly may not change.

Similarly, to determine an accurate value of oxygen consumption, measurements of Qᵢₙₕ, and thus Vᵢₙₕ, may be taken at a point that is representative of the flow rate, and thus volume, of inhaled gas at a point corresponding to the point at which the oxygen concentration measurement of the exhaled gas is taken. With reference to FIG. 1, if the first oxygen sensor 108 is configured to sample a portion of the second gas exhaled by the patient via a connection with the exhalation limb 104, then the flow rate sensor 112 may be configured to sample a portion of the first gas via a connection to the inhalation limb 102. If the first oxygen sensor 108 is configured to sample a portion of the second gas exhaled by the patient via a connection with the patient interface assembly 106, then the flow rate sensor 112 may be configured to sample a portion of the first gas via a connection with the patient interface assembly (or the flow rate sensor measurement may need to be adapted using information from the ventilator on the by-pass flow, as explained in more detail below).

In some examples, flow rate measurements and oxygen concentration measurements may be performed in different locations (e.g., one via connection with the exhalation limb and the other via a connection with the patient interface assembly). In this case, the oxygen concentration measurements and/or the flow rate measurements may be adjusted such that they correspond (e.g., as if they were performed at the same location). Similarly, measurements of carbon dioxide concentrations may be performed in a different location to the flow rate measurements and/or the oxygen concentration measurements. In this case, the carbon dioxide concentration measurements, oxygen concentration measurements and/or flow rate measurements may be adjusted such that they correspond (e.g., as if they were performed at the same location). Information relating to the bypass flow may be used to adjust measurements such that they correspond. For example, information relating to the bypass flow may allow a measurement of oxygen concentration of the second gas measured via a connection with the patient interface assembly to be adjusted such that it corresponds to the flow rate measurement as measured on the exhalation limb (e.g., as if the oxygen concentration of the second gas were measured at a point along the exhalation limb, or as if the flow rate measurement was measured at the patient interface assembly).

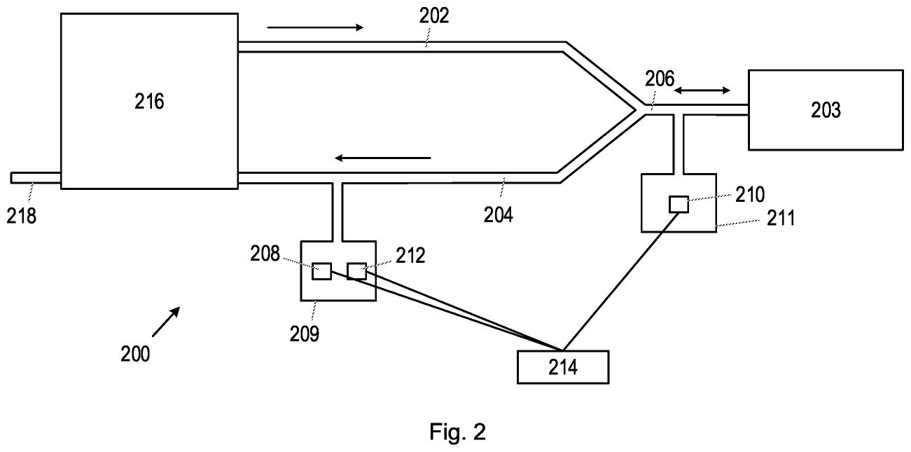

FIG. 2 is a schematic illustration of a further example of a system 200 for determining a level of oxygen consumption of a patient. The system 200 comprises an inhalation limb 202 configured to transport a first gas for inhalation by a patient 203, an exhalation limb 204 configured to transport a second gas exhaled by the patient, and a patient interface assembly 206 connected to the inhalation limb and the exhalation limb, the patient interface assembly to transport the first gas to the patient and the second gas from the patient. The system 200 further comprises a first oxygen sensor 208 configured to measure a concentration of oxygen in the second gas at a first sampling region 209. The system 200 further comprises a first carbon dioxide sensor 210 configured to measure a concentration of carbon dioxide in the second gas at a second sampling region 211. The system 200 further comprises a flow rate sensor 212 configured to measure the flow rate of the second gas at the first sampling region 209. The system 200 further comprises a processor 214 configured to determine a level of oxygen consumption of the patient using an indication of a concentration of oxygen in the first gas, an indication of a concentration of carbon dioxide in the first gas and data obtained via the first oxygen sensor, the first carbon dioxide sensor and the flow rate sensor. The system 200 further comprises a mechanical ventilator 216 configured to generate a flow of gas (e.g., the first gas) to the patient 203. and to vent gas transported away from the patient. The mechanical ventilator 216 comprises an exhaust 218.

In some examples, a mixing chamber may comprise the first sampling region 209. In some examples, the first sampling region may be configured to sample a portion of exhaled gas from the exhalation limb 204 (as shown in FIG. 2), from the exhaust 218 of the mechanical ventilator 216 (not shown), or from the patient interface assembly 206 (not shown).

FIG. 2 shows a specific example of a system for determining a level of oxygen consumption of a patient. components of the system shown in FIG. 2 may be positioned elsewhere, in accordance with embodiments disclosed herein.

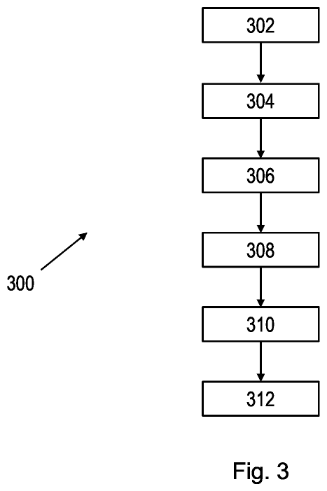

According to a second aspect, a method (e.g., a computer-implemented method) for determining a level of oxygen consumption of a patient is provided. FIG. 3 is a flowchart of an example of a computer-implemented method 300 for determining a level of oxygen consumption of a patient. In some examples, a processor may be configured to perform one or more steps of the method 300. The method comprises, at step 302, receiving first oxygen concentration data indicative of a concentration of oxygen in a first gas to be inhaled by a patient. In some examples, the first oxygen concentration data may be received from a mechanical ventilator. For example, the first oxygen concentration data may comprise an oxygen concentration setting of the mechanical ventilator (e.g., a pre-set value of oxygen concentration set by a user). In other examples, the first oxygen concentration data may comprise oxygen concentration data generated as a result of a measurement of a concentration of oxygen in the first gas measured by an oxygen sensor located within, or associated with, the mechanical ventilator. In some examples, the first oxygen concentration data may comprise oxygen concentration data generated as a result of a measurement of a concentration of oxygen in the first gas measured by an oxygen sensor located at a third sampling region. For example, the first oxygen concentration data may comprise oxygen concentration data measured using an oxygen sensor configured to sample a portion of the first gas in the inhalation limb or in the patient interface assembly. The oxygen sensor may be configured to measure a concentration of oxygen in the first gas indirectly (e.g., via a side-stream measurement). In some examples, the third sampling region may be located in a mixing chamber (e.g., the second mixing chamber).

The method 300 comprises, at step 304, receiving first carbon dioxide data indicative of a concentration of carbon dioxide in the first gas. The first carbon dioxide data may be received from the mechanical ventilator. For example, the mechanical ventilator may comprise a carbon dioxide sensor configured to measure a concentration of carbon dioxide in the first gas. In some examples, the first carbon dioxide data may be a predefined value (e.g., not measured). For example, the first carbon dioxide data may comprise a value of a concentration of carbon dioxide in the first gas corresponding to that of the carbon dioxide concentration of ambient air (e.g., 400 ppm, or the like). In some examples, the first carbon dioxide data may comprise a value of a concentration of carbon dioxide in the first gas of zero. Assuming a predefined value of a concentration of carbon dioxide in the first gas of zero can be justified in light of the relatively high concentration of carbon dioxide that may be generated by a patient during normal breathing. Advantageously, if the first carbon dioxide data is set at a predefined value (e.g., zero, 400 ppm, or the like), then a carbon dioxide sensor is not required to measure a concentration of carbon dioxide in the first gas, simplifying the system, or architecture, required to determine a level of oxygen consumption of a patient.

The method 300 comprises, at step 306, receiving second oxygen concentration data indicative of a concentration of oxygen in a second gas comprising gas exhaled by the patient, the concentration of oxygen of the second gas measured using an oxygen sensor at a first sampling region. In some examples, the second oxygen concentration data may comprise oxygen concentration data measured using an oxygen sensor configured to sample a portion of the second gas in the exhalation limb, in the patient interface assembly, or at the exhaust of a mechanical ventilator. For example, the oxygen sensor may be configured to sample the second gas via a connection with the patient interface assembly, via a connection with the exhalation limb, or via a connection with the exhaust of the mechanical ventilator. The oxygen sensor may be configured to measure a concentration of oxygen in the second gas indirectly (e.g., via a side-stream measurement).

The method 300 comprises, at step 308, receiving second carbon dioxide data indicative of a concentration of carbon dioxide in the second gas, the concentration of carbon dioxide in the second gas measured using a first carbon dioxide sensor at a second sampling region. In some examples, the first carbon dioxide sensor may comprise a capnography device. In some examples, the first carbon dioxide sensor may be configured to sample a portion of the second gas in the exhalation limb, in the patient interface assembly, or at the exhaust of a mechanical ventilator. For example, the first carbon dioxide sensor may be configured to sample the second gas via a connection with the patient interface assembly, via a connection with the exhalation limb, or via a connection with the exhaust of the mechanical ventilator. The first carbon dioxide sensor may be configured to measure a concentration of carbon dioxide in the second gas directly (e.g., in-situ) or indirectly (e.g., via a side-stream measurement).

The method 300 comprises, at step 310, receiving flow rate data indicative of a flow rate of the first gas or the second gas. The flow rate of the second gas may be measured using any known flow rate sensor known to the skilled person. The flow rate sensor may be configured to measure a flow rate of the second gas indicative of the flow rate of the second gas at the first sampling region. For example, the flow rate sensor may be located upstream or downstream of the first sampling region. In some examples, the first sampling region may comprise a region located within a first mixing chamber. In some examples, the flow rate sensor configured to measure a flow rate of the second gas at the first sampling location and the and the oxygen sensor configured to measure a concentration of oxygen in the second gas at the first sampling location may be located within the first mixing chamber. In some examples, the flow rate sensor may be located upstream or downstream of the first mixing chamber. For example. the flow rate sensor may be located on an inlet or an outlet of the first mixing chamber, may be configured to sample the second gas via connection with the exhalation limb upstream or downstream of the first mixing chamber, or the like. In some examples, the flow rate sensor may be located within the first mixing chamber (e.g., between the inlet and outlet of the first mixing chamber). The method 300 comprises, at step 312, determining, based on the received data, a level of oxygen consumption of the patient. In some examples, the received data may be received by a processor (e.g., the processor 114). In some examples, the processor may be configured to receive data from the mechanical ventilator, one or more of the oxygen sensors and/or one or more of the carbon dioxide sensors directly. For example, a processor associated with the mechanical ventilator, one or more of the oxygen sensors and/or one or more of the carbon dioxide sensors may be configured to transmit data to the processor 114. In other examples, data associated with (e.g., generated by) the mechanical ventilator (or a sensor located within, or associated with, the mechanical ventilator), one or more of the oxygen sensors and/or one or more of the carbon dioxide sensors may be configured to send data to an intermediate location, such as a server located in a cloud-computing environment. A level of oxygen consumption of a patient may be determined using a processor in located on a remote server, such as a processor located in such a cloud-computing environment.

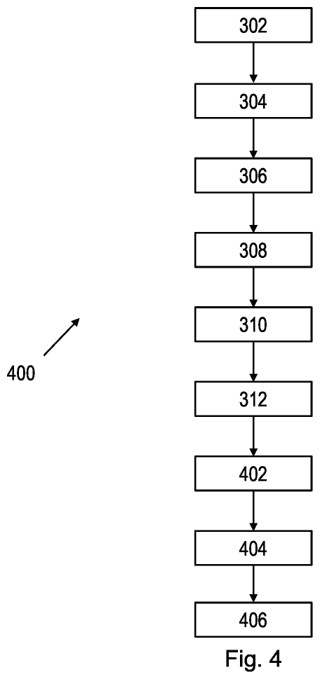

FIG. 4 is a flowchart of an example of a computer-implemented method 300 for determining a level of oxygen consumption of a patient. In some examples, a processor may be configured to perform one or more steps of the method 400. The method 400 may comprise one or more of the steps of method 300. In some embodiments, the method 400 may comprise, at step 402, receiving third carbon dioxide data indicative of a concentration of carbon dioxide in the second gas measured using a second carbon dioxide sensor located at the first sampling location.

The method 400 may comprise, at step 404, comparing the second carbon dioxide data with the third carbon dioxide data.

The method 400 may comprise, at step 406, generating an alert responsive to determining that a difference between the second carbon dioxide data and the third carbon dioxide data exceeds a threshold level. A difference between the second carbon dioxide data and the third carbon dioxide data may arise due to a fault in the second carbon dioxide data and/or the third carbon dioxide data. Advantageously, a fault may therefore be identified in the first carbon dioxide sensor and/or the second carbon dioxide sensor. Using a second carbon dioxide sensor to verify a measurement of the first carbon dioxide sensor may highlight when the first carbon dioxide sensor is faulty.

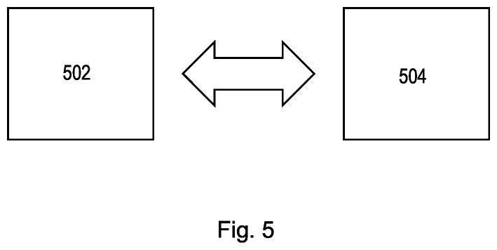

According to a third aspect, a computer program product is provided. FIG. 5 is a schematic illustration of a non-transitory computer readable medium 502 in communication with a processor 504. In some embodiments, a computer program product comprising a non-transitory computer readable medium 502 may be provided, the computer readable medium having computer readable code embodied therein, the computer readable code being configured such that, on execution by a suitable computer or processor 504, the computer or processor is caused to perform steps of the methods disclosed herein.

The processor 114, 504 can comprise one or more processors, processing units, multi-core processors or modules that are configured or programmed to control components of the system 100 in the manner described herein. In particular implementations, the processor 114, 504 can comprise a plurality of software and/or hardware modules that are each configured to perform, or are for performing, individual or multiple steps of the method described herein.

The term “module”, as used herein is intended to include a hardware component, such as a processor or a component of a processor configured to perform a particular function, or a software component, such as a set of instruction data that has a particular function when executed by a processor.

It will be appreciated that the embodiments of the invention also apply to computer programs, particularly computer programs on or in a carrier, adapted to put the invention into practice. The program may be in the form of a source code, an object code, a code intermediate source and an object code such as in a partially compiled form, or in any other form suitable for use in the implementation of the method according to embodiments of the invention. It will also be appreciated that such a program may have many different architectural designs. For example, a program code implementing the functionality of the method or system according to the invention may be sub-divided into one or more sub-routines. Many different ways of distributing the functionality among these sub-routines will be apparent to the skilled person. The sub-routines may be stored together in one executable file to form a self-contained program. Such an executable file may comprise computer-executable instructions, for example, processor instructions and/or interpreter instructions (e.g. Java interpreter instructions). Alternatively, one or more or all of the sub-routines may be stored in at least one external library file and linked with a main program either statically or dynamically, e.g. at run-time. The main program contains at least one call to at least one of the sub-routines. The sub-routines may also comprise function calls to each other. An embodiment relating to a computer program product comprises computer-executable instructions corresponding to each processing stage of at least one of the methods set forth herein. These instructions may be sub-divided into sub-routines and/or stored in one or more files that may be linked statically or dynamically. Another embodiment relating to a computer program product comprises computer-executable instructions corresponding to each means of at least one of the systems and/or products set forth herein. These instructions may be sub-divided into sub-routines and/or stored in one or more files that may be linked statically or dynamically.

The carrier of a computer program may be any entity or device capable of carrying the program. For example, the carrier may include a data storage, such as a ROM, for example, a CD ROM or a semiconductor ROM, or a magnetic recording medium, for example, a hard disk. Furthermore, the carrier may be a transmissible carrier such as an electric or optical signal, which may be conveyed via electric or optical cable or by radio or other means. When the program is embodied in such a signal, the carrier may be constituted by such a cable or other device or means. Alternatively, the carrier may be an integrated circuit in which the program is embedded, the integrated circuit being adapted to perform, or used in the performance of, the relevant method.

Variations to the disclosed embodiments can be understood and effected by those skilled in the art in practicing the principles and techniques described herein, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored or distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A system for determining a level of oxygen consumption of a patient, comprising:
an inhalation limb configured to transport a first gas for inhalation by a patient;
an exhalation limb configured to transport a second gas comprising gas exhaled by the patient;
a patient interface assembly connected to the inhalation limb and the exhalation limb, the patient interface assembly to transport the first gas to the patient and the second gas from the patient;
a first oxygen sensor configured to measure a concentration of oxygen in the second gas at a first sampling region;
a first carbon dioxide sensor configured to measure a concentration of carbon dioxide in the second gas at a second sampling region;
a flow rate sensor configured to measure a flow rate of the first gas or the second gas; and
a processor configured to determine a level of oxygen consumption of the patient using an indication of a concentration of oxygen in the first gas, an indication of a concentration of carbon dioxide in the first gas and data obtained via the first oxygen sensor, the first carbon dioxide sensor and the flow rate sensor.

2. A system according to claim 1, further comprising a first mixing chamber configured to receive a portion of the second gas, the first mixing chamber comprising one or more of:
the first oxygen sensor; and
a second carbon dioxide sensor configured to measure a concentration of carbon dioxide in the portion of the second gas to be used for verification of the concentration of carbon dioxide measured using the first carbon dioxide sensor.

3. A system according to claim 2, wherein the first mixing chamber is configured to receive a portion of the second gas via a connection with the patient interface assembly or via a connection with the exhalation limb.

4. A system according to claim 2, further comprising:
a mechanical ventilator;
wherein the inhalation limb and the exhalation limb are connected to the mechanical ventilator, and wherein one or more of the following conditions are satisfied:
(a) the first mixing chamber is configured to receive a portion of the second gas via a connection with an exhaust of the mechanical ventilator;
(b) the concentration of oxygen in the first gas is determined using the mechanical ventilator; and
(c) the concentration of carbon dioxide in the first gas is determined using the mechanical ventilator.

5. A system according to claim 1, further comprising one or more of:
(a) a second oxygen sensor configured to measure the concentration of oxygen in the first gas at a third sampling region; and
(b) a third carbon dioxide sensor configured to measure the concentration of carbon dioxide in the first gas at a fourth sampling region.

6. A system according to claim 5, further comprising a second mixing chamber configured to receive a portion of the first gas via a connection with the inhalation limb or with the patient interface assembly, the second mixing chamber comprising one or more of the second oxygen sensor and the third carbon dioxide sensor.

7. A system according to claim 1, wherein the first carbon dioxide sensor is a capnography device.

8. A system according to claim 7, wherein the capnography device is a mainstream capnography device or a sidestream capnography device.

9. A system according to claim 1, further comprising a patient y-connector comprising a first port connected to an end of the inhalation limb, a second port connected to an end of the exhalation limb and a third port connected to an end of the patient interface assembly.

10. A computer-implemented method for determining a level of oxygen consumption of a patient, comprising:
receiving first oxygen concentration data indicative of a concentration of oxygen in a first gas to be inhaled by a patient;
receiving first carbon dioxide data indicative of a concentration of carbon dioxide in the first gas;
receiving second oxygen concentration data indicative of a concentration of oxygen in a second gas comprising gas exhaled by the patient, the concentration of oxygen of the second gas measured using an oxygen sensor at a first sampling region;
receiving second carbon dioxide data indicative of a concentration of carbon dioxide in the second gas, the concentration of carbon dioxide in the second gas measured using a first carbon dioxide sensor at a second sampling region;
receiving flow rate data indicative of a flow rate of the first gas or the second gas; and
determining, based on the received data, a level of oxygen consumption of the patient.

11. A method according to claim 10, further comprising:
receiving third carbon dioxide data indicative of a concentration of carbon dioxide in the second gas measured using a second carbon dioxide sensor located at the first sampling location;
comparing the second carbon dioxide data with the third carbon dioxide data; and
generating an alert responsive to determining that a difference between the second carbon dioxide data and the third carbon dioxide data exceeds a threshold level.

12. A method according to claim 10, wherein determining a level of oxygen consumption, VO2, of the patient comprises using the formula
$$\frac{VCO 2}{{V}_{exh}} or \frac{VCO 2}{{V}_{inh}} ,$$
wherein FiO2 is a concentration of oxygen in the first gas, FiCO2 is a concentration of carbon dioxide in the first gas, FeO2 is a concentration of oxygen in the second gas, FeCO2 is a concentration of carbon dioxide in the second gas, Vₑₓₕ is the average flow rate of the second gas at the point of measurement of the concentration of oxygen in the second gas, and Vᵢₙₕ is the average flow rate of the first gas, and wherein FeCO2 is determined using the formula
$$VO 2 = {V}_{exh} ( FiO 2 * \frac{( 1 - FeO 2 - FeCO 2 )}{( 1 - FiO 2 - FiCO 2 )} - FeO 2 ) or VO 2 = {V}_{inh} ( FiO 2 - FeO 2 * \frac{( 1 - FiO 2 - FiCO 2 )}{( 1 - FeO 2 - FeCO 2 )} ) ,$$
wherein VCO2 is a value of carbon dioxide production determined using the second carbon dioxide data.

13. A method according to claim 12, wherein each of the variables Vₑₓₕ, FiO2, FeO2, FiCO2 and FeCO2 represents an average value over a defined time period.

14. A method according to claim 10, wherein first carbon dioxide data comprises an ambient carbon dioxide concentration.

15. A computer program product comprising a non-transitory computer readable medium, the computer readable medium having computer readable code embodied therein, the computer readable code being configured such that, on execution by a suitable computer or processor, the computer or processor is caused to perform the method of claim 10.
